(19) United States
(12) Patent Application Publication
(10) Pub. No.: US 2025/0235470 A1
(43) Pub. Date: Jul. 24, 2025

(54) Methods and Compositions for Inhibiting the Growth and/or proliferation of MYC-Driven Tumor Cells

(71) Applicant: Fred Hutchinson Cancer Center, Seattle, WA (US)

(72) Inventors: Masafumi TOYOSHIMA, Sendai (JP); Carla GRANDORI, Seattle, WA (US)

(21) Appl. No.: 18/661,284

(22) Filed: May 10, 2024

Related U.S. Application Data
(63) Continuation of application No. 18/479,773, filed on Oct. 2, 2023, now abandoned.
Application No. 18/479,773 is a continuation of application No. 18/161,753, filed on Jan. 30, 2023, now abandoned.
Application No. 18/161,753 is a continuation of application No. 17/738,964, filed on May 6, 2022, now abandoned.
Application No. 17/738,964 is a continuation of application No. 17/489,517, filed on Sep. 29, 2021, now abandoned.
Application No. 17/489,517 is a continuation of application No. 17/174,124, filed on Feb. 11, 2021, now abandoned.
Application No. 17/174,124 is a continuation of application No. 16/919,484, filed on Jul. 2, 2020, now abandoned.
Application No. 16/919,484 is a continuation of application No. 16/689,957, filed on Nov. 20, 2019, now abandoned.
Application No. 16/689,957 is a continuation of application No. 15/604,993, filed on May 25, 2017, now abandoned.
Application No. 15/604,993 is a continuation of application No. 15/000,933, filed on Jan. 19, 2016, now abandoned.
Application No. 15/000,933 is a continuation of application No. 14/237,838, filed on Jun. 12, 2014, now Pat. No. 9,273,317.
(60) Provisional application No. 61/521,715, filed on Aug. 9, 2011.

(51) Int. Cl. A61K 31/65; A61K 31/00; A61K 31/404; A61K 31/713; C12N 15/113

(52) U.S. Cl. CPC A61K 31/65; A61K 31/00; A61K 31/404; A61K 31/713; C12N 15/1135; C12N 15/1137; C12Y 207/11001; C12N 2310/14

(57) ABSTRACT

The invention generally relates to methods for identifying and using anticancer therapeutic agents and, more particularly, to methods for identifying and using inhibitors of genes for inhibiting the growth and/or proliferation of MYC-driven tumor cells relative to normal cells.

### CROSS-REFERENCE TO RELATED APPLICATIONS

This application is a Continuation of U.S. application Ser. No. 18/479,773, filed Oct. 2, 2023, which is a Continuation of U.S. application Ser. No. 18/161,753, filed Jan. 30, 2023, which is a Continuation of U.S. application Ser. No. 17/738,964, filed May 6, 2022, which is a Continuation of U.S. application Ser. No. 17/489,517, filed Sep. 29, 2021, which is a Continuation of U.S. application Ser. No. 17/174,124, filed Feb. 11, 2021; which is a Continuation of U.S. application Ser. No. 16/919,484, filed Jul. 2, 2020; which is a Continuation of U.S. application Ser. No. 16/689,957, filed Nov. 20, 2019; which is a Continuation of U.S. application Ser. No. 15/604,993, filed May 25, 2017; which is a Continuation of U.S. application Ser. No. 15/000,933, filed Jan. 19, 2016; which is a Continuation of U.S. application Ser. No. 14/237,838, filed Jun. 12, 2014, now U.S. Pat. No. 9,273,317, issued Mar. 1, 2016; which is a 371 National Phase Application of International Application No. PCT/US2012/050186, filed Aug. 9, 2012; which claims the benefit under 35 U.S.C. § 119 (e) of U.S. Provisional Patent Application 61/521,715, filed Aug. 9, 2011, each of which is incorporated by reference herein in its entirety.

### STATEMENT OF GOVERNMENT LICENSE RIGHTS

This invention was made with Government support under AG026661 awarded by the National Institutes of Health. The Government has certain rights in the invention.

### STATEMENT REGARDING THE SEQUENCE LISTING

The Sequence Listing XML associated with this application is provided in XML file format and is hereby incorporated by reference into the specification. The name of the XML file containing the Sequence Listing XML is FHCR_014_11US_ST26.xml. The XML file is 479,629 bytes, and created on May 9, 2024, and is being submitted electronically via U.S. Patent Center.

### FIELD OF THE INVENTION

The invention relates generally to methods for identifying and using anticancer therapeutic agents and, more particularly, to methods for identifying and using inhibitors of genes for inhibiting the growth and/or proliferation of MYC-driven tumor cells relative to normal cells.

### BRIEF SUMMARY

Embodiments of the present invention include methods for inhibiting the growth and/or proliferation of a myc-driven cancer or tumor cell comprising the step of contacting the cancer or tumor cell with at least one inhibitor that inhibits the gene function of at least one of the genes listed in Table 1 or 2. In certain embodiments, the myc-driven cancer cell is derived from one of the following: a neuroblastoma tumor, a metastatic neuroblastoma tumor, a medulloblastoma, a lymphoma, a rhabdomyosarcoma, a melanoma, a lung cancer, a liver cancer, a breast cancer, a colon cancer, a prostate cancer, an ovarian cancer, or Burkitt's lymphoma.

In particular embodiments, the tumor cell is contacted in vitro. In specific embodiments, the cancer cell is contacted in vivo in a mammalian subject, optionally a human patient diagnosed with a MYC-driven cancer, such as any of the aforementioned cancers/tumors.

In some embodiments, the inhibitor is a small molecule inhibitor that inhibits the function of the gene product. In certain embodiments, the inhibitor interferes with the transcription of mRNA from the gene. In particular embodiments, the inhibitor interferes with production/expression of functional gene product of the gene.

In certain embodiments, the gene is selected from the genes listed in Table 1. In specific embodiments, the gene is selected from the group consisting of ALDOA, CECR2, IGF2R, PAK6, PES1, RAD21, REV1L, SUV39H1, TIE1.

Also included are methods of treating a subject suffering from a tumor comprising myc-driven tumor cells, comprising administering to the subject an amount of a composition comprising an inhibitor that inhibits the gene function, transcription, production/expression, or activity of the gene product of at least one of the genes listed in Table 1 or 2, and is effective to inhibit the growth and/or proliferation of the tumor cells.

### BRIEF DESCRIPTION OF THE DRAWINGS

FIG. 1. Identification of synthetic lethal genes with c-MYC overexpression by high throughput siRNA screening. a, Graphical schematic of the siRNA screen. HFF expressing either c-MYC or transduced with an empty retroviral vector, pB, were plated in 384 well plates. 24 hr later they were transfected with siRNA pools (3 siRNAs/pool) targeting a total of ˜3,311 genes, with one gene targeted per well. At day 5, the viability was quantified utilizing an Alamar Blue staining assay and an En Vision plate reader. b, Graphical representation of cell viability as affected by each siRNA pool in both HFF-pB or HFF-c-MYC and quantified as % relative to mock (on a log 10 scale). Values represent the average viability of 3 replicates. A set of siRNAs (targeting 102 genes labeled as light shaded+) caused differential loss of viability (Z score>2) in the HFF-MYC versus control pB. An additional set of siRNAs targeting 20 genes caused selective growth advantage to HFF-MYC. c, CSNK1e, PES1 and CECR2 mRNA levels following lentiviral-mediated shRNA knock-down. Relative levels of each gene were calculated using the ΔΔCT method and using GAPDH to normalize mRNA levels within each sample. d, Viability of HFF-pBabe and HFF-Myc following stable, lentiviral based knock-down of MYC-SL genes CSNK1e, PES1 and CECR2. Values represent mean viability from 3 independent assays. e, -γ-H2AX staining following transduction of HFF-pBabe and HFF-MYC with siRNAs pools corresponding to 40 MYC-SL genes and a previously identified MYC transcriptional target, DDX18. Representative images of anti-γ-H2AX staining from the INCell automated scope (20× magnification) are shown. f, Graphical representation of the γ-H2AX staining shown in (e). Y-axis indicates the % of cells stained with anti-γ-H2AX that scored for nuclear fluorescence levels above a negative control established threshold. Quantitation was obtained by automated microscopy in 96 well format, from triplicate samples. g, Quantitative assessment of Caspase-3 and -7 cleavage following transfection of the same siRNA pools as above (measured by the CaspaseGlo kit, Promega). Horizontal dark bold line indicates the background levels in HFF c-Myc cells. Results were normalized for cell number by the Alamar Blue assay.

FIG. 2. The knockdown of CSNK1e in neuroblastoma cell lines impairs growth of neuroblastoma with MYCN amplification in vitro and in vivo. a, Relative levels of CSNK1e mRNA following doxycycline or DMSO treatment of neuroblastoma cells harboring shCSNK1e #1, #2 and sh control expressing lentivirus. Relative levels of each gene were calculated using the ΔΔCT method and using GAPDH to normalize mRNA levels within each sample. b, Viability assessment following growth under doxycycline containing medium for 4 days as measured by CellTiter Glo (Promega). Values represent mean viability normalized to mock treated cells. c, Representative western blot showing levels of CSNK1e protein in neuroblastoma cells transduced with doxycycline inducible shRNA lentivirus (two different sh: #1 and #2) targeting CSNK1e and non-target sequence (shControl) used in FIG. 2a. Cells were cultured in the presence or absence of doxycycline for 4 days. Actin is shown as a loading control. d, Xenograft tumor growth of SK-N-BE2 neuroblastoma cells transduced with doxycycline inducible shRNA for CSNK1e or shControl in NOD/SCID mice. Doxycycline exposure was started when tumors reached a size of about 100 mm³. Knockdown of CSNK1e inhibited growth of established xenograft in 3 out of 4 mice compared to no doxycycline treated control (arrows).

FIG. 3. IC261 treatment blocks MYCN amplified neuroblastoma tumor growth in vivo. a, Representative images of MYCN amplified neuoblastoma xenograft in NOD/SCID mice before and after treatment with either DMSO or IC261. Tumors were engrafted, and allowed to reach a size of about 100 mm³, then IC261 (21.5 mg/kg) or DMSO was injected subcutaneously daily for 8 days. b, Quantitation of tumor size over the 8-day treatment regimen with either IC261 or DMSO control. Values represent mean tumor volume at each time point (n=5 for each group, error bars indicate SD). c, Immunohistochemical analysis of tumor sections from IC261 and DMSO treatment groups described in a and b. Representative images of H-E, TUNEL and BrdU staining for each group are shown. BrdU was administered 2h before collection. d. Quantification of TUNEL positive cells and BrdU positive cells per field in DMSO or 1C-261 treated xenograft tumors. Error bars indicate SD of means.

FIG. 4. CSNK1e expression correlates with poor prognosis and MYCN amplification in neuroblastoma. a, Kaplan-Meier survival curves of neuroblastoma patients divided on the basis of CSNK1e expression; lower solid line indicates high and upper solid line indicates low CSNK1e mRNA expression based on microarray data accessible at the Oncogenomics neuroblastoma prognosis database. b, Graphical representation of expression intensities for CSNK1e mRNA derived from microarray data of neuroblastoma tumor samples. Each bar represents one sample. The shaded horizontal line (-MYCN ampl.) indicates samples derived from MYCN amplified neuroblastoma c. Representative western blot of CSNK1e, MYCN and β-actin (loading control) protein levels in SN-N-AS (MYCN not amplified), SK-N-BE2 and IMR-32 (MYCN amplified) neuroblastoma cells. d, Representative western blot of CSNK1e, MYCN and β-actin (loading control) protein levels in HFF pB and HFF c-Myc cells. e, Representative western blot of CSNK1c, MYCN and β-actin (loading control) protein levels in Tet21N (MYCN Tet-Off) cells. f, Real time RT-PCR quantification of the relative levels of each casein kinase 1 isoforms normalized to glyceraldehydes-3-phosphate dehydrogenase (GAPDH) mRNA levels in neuroblastoma cell lines with or without MYCN amplification. The bars for each isoform, from left to right, refer to SK-N-AS, SH-SY-5Y, LAN-5, IMR-32, KCN, KCNR, SK-N-BE2 neuroblastoma cell lines.

FIG. 5. Network analysis of MYC-SL “Hits” (light shading) and their connection with a pre-assembled MYC “core” pathway (dark shading). The network was built to visualize known literature connections between the “Hits” (light shading) and a pre-assembled MYC core pathway (dark shading). All connections were drawn based on Ingenuity curated database. Only the MYC-SL with known direct connections with the MYC core components are here visualized. Each line represents a single reference and the connecting lines indicate the type of interaction as indicated in the box. Arrows mark genes referred to in the text.

FIG. 6. Conditional knock-down of CSNK1e with lentiviral expressed short hairpins docs not affect expression of other CSNK1e isoforms.

Relative mRNA expression of CSNK1 A (α), 01 (γ11), G2 (γ2), G3 (γ3) and D (8) in SKNBE2 cells were transduced with lentiviral vectors expressing shCSNK1e #1 and #2 (see FIG. 2) and either treated or untreated with Doxycycline for 48 hrs. Relative levels of each gene were calculated using the ΔΔCT method and using GAPDH to normalize mRNA levels within each sample.

FIG. 7. Chemical inhibition of CSNK1e kinase activity shows selective toxicity to MYC overexpressing cells.

a. HFF cell lines with or without c-Myc over-expression were treated with 0-10 uM 1C261 for 48 hrs. The cells were exposed to CellTiter-Glo reagent and viability was assessed by ATP-induced chemiluminescence. Values indicate mean±SD. b. Tct21N cells with or without doxycyclineatment and IMR-32 cells (MYCN+) were treated with 0-30 uM 1C261 for 48 hrs. The cells were exposed to CellTiter-Glo reagent and the viability was assessed by ATP-induced chemiluminescence. Values indicate mean±SD. c. Cell growth curves for HFF-pBabe incubated with different concentrations of 1C261. d. Cell growth curves for HFF-MYC incubated with different concentrations of 1C261.

FIG. 8. The CSNK1e gene contains MYC-MAX consensus sites. The DNA sequence surrounding the transcription start site as well as the first and second intron of CSNK1e contains several MYC-MAX potential binding sites 41 both upstream and downstream from the transcription start site.

### DETAILED DESCRIPTION

Embodiments of the present invention relate to the discovery of druggable gene targets in MYC-driven cancers, and related methods of inhibiting the growth and/or proliferation of myc-driven cancer cells by targeting one or more of these genes or their encoded protein(s) with inhibitory agent(s), including small molecule inhibitors of the protein(s). Also included are methods using such inhibitors to treat a subject having a MYC-driven cancer. In particular aspects, the cancer is a c-MYC-driven or a MYCN-driven cancer, such as a c-MYC-amplified or a MYCN-amplified cancer, and the gene (or its encoded protein) targeted for inhibition is described in Table 1 or 2.

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

“Cancer” relates generally to a class of diseases or conditions in which a group of cells display one or more of uncontrolled growth (i.e., division beyond normal limits), invasion (i.e., intrusion on and destruction of adjacent tissues), and/or metastasis (i.e., spread to other locations in the body via lymph or blood). These malignant properties of cancers differentiate them from benign cancers, which are self-limited, and typically do not invade or metastasize.

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancers form solid tumors, but some, e.g., leukemia, do not necessarily form tumors. For those cancers that form tumors, the terms cancer (cell) and tumor (cell) are used interchangeably.

As used herein, the terms “function” and “functional” and the like refer to a biological, enzymatic, or therapeutic function.

The term “gene” refers to a locatable region of genomic sequence, corresponding to a unit of inheritance, which is associated with regulatory regions, transcribed regions, and or other functional sequence regions. A gene optionally encodes for a protein or polypeptide that has at least one function in an organism.

The terms “modulating” and “altering” include “increasing,” “enhancing” or “stimulating,” as well as “inhibiting,” “decreasing” or “reducing,” typically in a statistically significant or a physiologically significant amount or degree relative to a control. An “increased,” “stimulated” or “enhanced” amount is typically a “statistically significant” amount, and may include an increase that is 1.1, 1.2, 2, 3, 4, 5, 6, 7, 8, 9, 10, 15, 20, 30 or more times (e.g., 500, 1000 times) (including all integers and decimal points in between and above 1, e.g., 1.5, 1.6, 1.7, 1.8, etc.) the amount produced by no composition (e.g., the absence of polypeptide of conjugate of the invention) or a control composition, sample or test subject. A “decreased” or “reduced” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease in the amount produced by no composition or a control composition, including all integers in between. As one non-limiting example, a control could compare the growth and/or proliferation of a MYC-driven tumor cell after being contacted with an inhibitor that inhibits the gene function of a gene Listed in Table 1 or 2, relative to the growth and/or proliferation of a normal/healthy of the same or similar type, or relative to the growth and/or proliferation of a non-MYC-driven tumor of the same or similar type, after being contacted with that same inhibitor. Other examples of comparisons and “statistically significant” amounts will be apparent to persons skilled in the art from the description provided herein.

The term “MYC” refers to the Myc family of transcription factors, including c-MYC (encoded by the MYC gene) and N-MYC (or MYCN; encoded by the MYCN gene). A “MYC-driven” cancer cell or cancer cell derived therefrom includes a cancer cell that has increased expression and/or activity of at least one Myc transcription factor such as c-MYC and/or MYCN, relative to a control cell such as a normal (e.g., non-cancerous) cell of the same or corresponding cell type. As one example, a “MYC-driven” cancer cell includes a “MYC-amplified” or “MYCN-amplified” cancer cell, such as a cell that has an increase (1.5×, 2×, 3×, 4×, etc.) in the number of copies (e.g., 1, 2, 3, 4, 5, 6 copies) of a MYC and/or a MYCN gene, optionally without a proportional increase in other genes.

By “statistically significant,” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

A “subject,” as used herein, includes any animal that has a cancer or exhibits a symptom or cancer, or is at risk for having a cancer or exhibiting a symptom of cancer, which can be treated by inhibiting the function of a gene described herein (see Table 1 and Table 2). Suitable subjects (patients) include laboratory animals (such as mouse, rat, rabbit, or guinea pig), farm animals, and domestic animals or pets (such as a cat or dog). Non-human primates and, preferably, human patients, are included. In certain aspects, prior to treatment with an inhibitor described herein, a subject is first identified as having a MYC-driven cancer or tumor, for instance, by measuring the expression levels and/or number of gene copies of a Myc transcription factor, such as MYC and/or MYCN. In some aspects, the subject is monitored before, during, and/or after treatment for the presence of a MYC-driven cancer or tumor, and the treatment is adapted accordingly.

“Substantially” or “essentially” means nearly totally or completely, for instance, 95%, 96%, 97%, 98%, 99% or greater of some given quantity.

“Treatment” or “treating,” as used herein, includes any desirable effect on the symptoms or pathology of a disease or condition such as a MYC-driven cancer, and may include even minimal changes or improvements in one or more measurable markers of the disease or condition being treated. “Treatment” or “treating” does not necessarily indicate complete eradication or cure of the disease or condition, or associated symptoms thereof. The subject receiving this treatment is any subject in need thereof. Exemplary markers of clinical improvement will be apparent to persons skilled in the art.

The term “wild-type” refers to a gene or gene product that has the characteristics of that gene or gene product when isolated from a naturally-occurring source. A wild type gene or gene product (e.g., a polypeptide) is that which is most frequently observed in a population and is thus arbitrarily designed the “normal” or “wild-type” form of the gene.

### Inhibition and Treatment of MYC-Driven Cancers

Drugs directed toward oncoproteins have demonstrated therapeutic efficacy while avoiding systemic toxicities associated with standard chemotherapeutics. However, the MYC family of oncoproteins, which are broadly implicated in many human cancers, are difficult to inhibit with small molecules or antibody based therapies. To target MYC-driven cancers, we have taken the approach of identifying druggable genes that exhibit a synthetic lethal relationship with aberrant MYC expression. Using an isogenic cell model system, we identified, via high throughput siRNA screening, more than 100 druggable genes that exhibit a synthetic lethal interaction with MYC (referred to as MYC-synthetic lethal genes, MYC-SL). Among the MYC-SL genes, we focused on casein kinase 1 epsilon (CSNK1e), whose relevance in MYC-driven human cancer was demonstrated by correlation between high levels of CSNK1e expression, MYCN amplification, and poor clinical prognosis in neuroblastoma cases. The requirement of CSNK1e for growth of neuroblastomas with MYCN amplification was validated in vivo by conditional knock-down and via a small molecule inhibitor of its activity. Thus, our studies show how high throughput siRNA screening can be used to identify a network of synthetic lethal genes and potential new therapeutic targets functionally linked to a previously un-druggable oncogene.

The MYC oncogene is a central driver in many human cancers, and its amplification is associated with poor prognosis in breast^{1 2} prostate^{3 4}, colon⁵ and pediatric cancers such as neuroblastoma (for review see⁶). In addition, c-MYC overexpression, together with gene amplification has been reported in over 50% of ovarian cancers⁷, in ˜30% of hepatocellular carcinoma⁸, and in a high percentage of small-cell and non-small-cell lung cancer⁹. Such a high frequency of MYC family deregulation in human cancers suggests that a strategy to target MYC-driven cancers may be relevant for the treatment of a broad population of patients. Recently, systemic inhibition of MYC utilizing a transgenic mouse model has demonstrated the efficacy of a dominant negative MYC in mediating tumor regression¹⁰. However, MYC family members encode for transcription factors without obvious druggable domains¹¹ rendering the identification of small molecule inhibitors a challenge¹². In addition, as MYC oncoproteins carry out essential functions in proliferative tissues¹³, prolonged inhibition of MYC function could cause severe side effects. Rather than targeting MYC itself, we elected to apply a functional genomic screen to identify druggable genes that are preferentially required for survival of MYC overexpressing cells. To avoid the genetic noise inherent in cancer cells, we chose to screen an isogenic pair of primary cells, where the only perturbation was overexpression of c-MYC through a retroviral vector¹⁴. Human foreskin fibroblasts (HFFs) are unique in that they do not senesce in response to MYC overexpression¹⁴ or activated Ras ¹⁵, a property that has been attributed to lack of culture stress. Furthermore, c-MYC overexpression in HFFs recapitulates both the gene expression signature and cellular phenotypes of MYC-driven cancers (^{14 16 17} and CG unpublished observations).

siRNA Screening to Identify a Network of Genes Required for Survival of c-MYC Overexpressing Cells

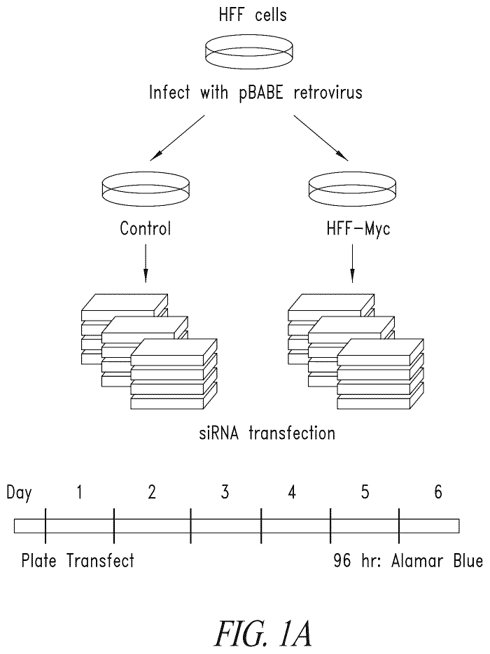

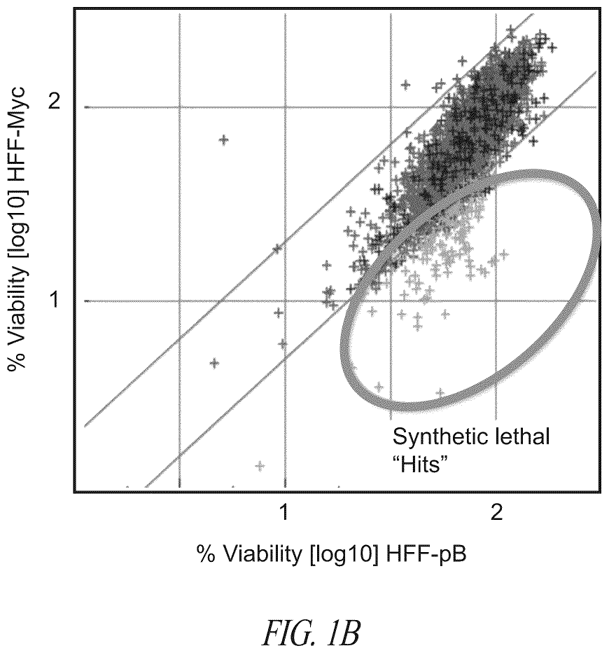

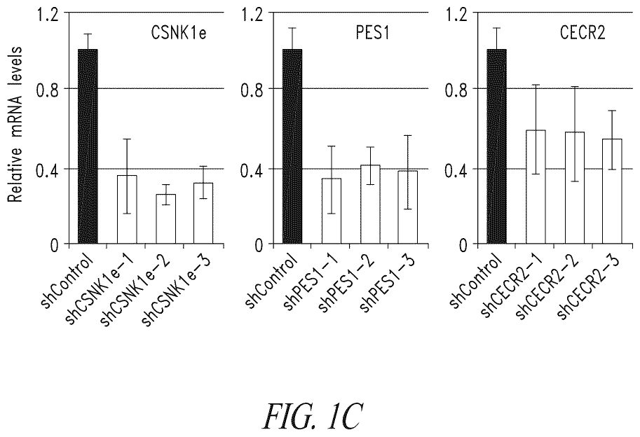

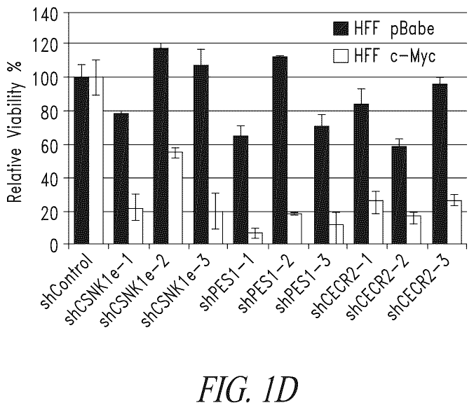

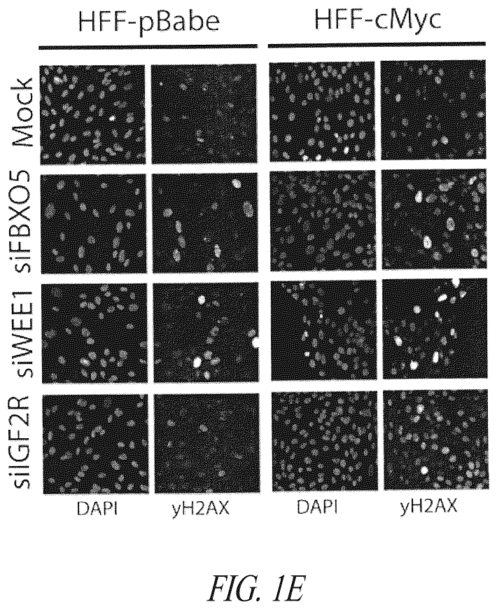

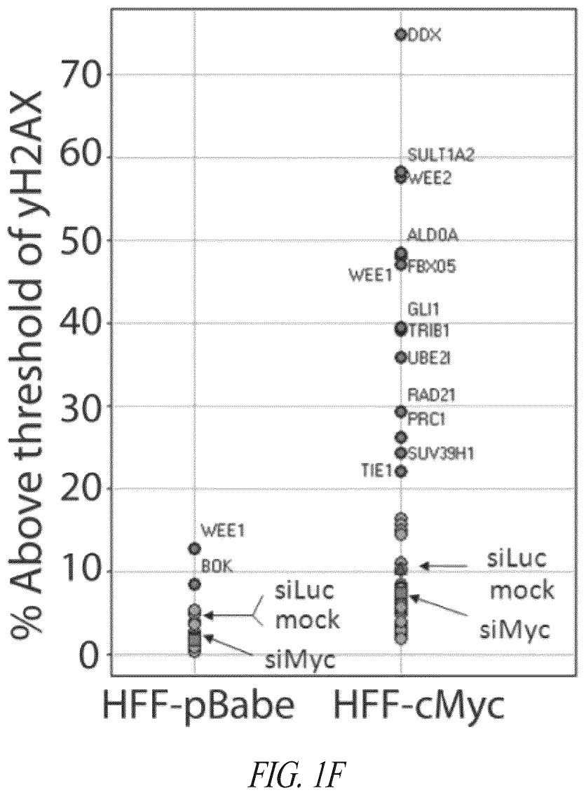

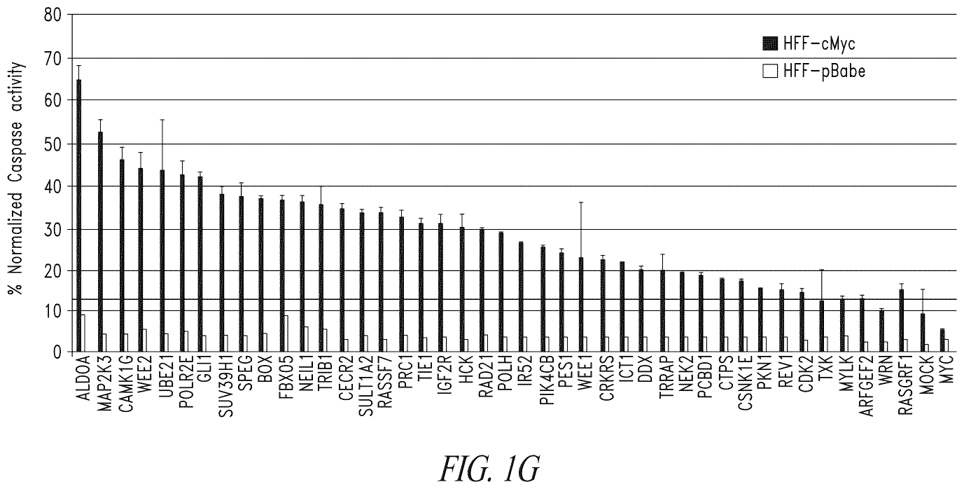

We employed a high throughput robotics-based approach for massive parallel testing of an arrayed siRNA library to accurately quantify the effects of siRNAs against ˜3,300 druggable genes and 200 microRNAs on the viability of HFF-MYC (stably transduced with a retroviral vector expressing c-MYC), or a control empty vector, HFF-pB¹⁴, see FIG. 1a for schematic of the experimental set-up). The siRNA library collection was selected so as to target all known human kinases, ubiquitin ligases, DNA repair proteins and a custom collection of genes involved in cancer pathways, with each target gene being interrogated by a pool of three unique siRNAs (SIGMA and Rosetta-Merck custom collection). Three technical replicates and the one gene/well approach enabled derivation of hits with statistical significance for each gene tested, similarly to what has been shown for other biological systems^{18 19}. Cell viability was assessed using Alamar Blue staining, and quantified using an EnVision plate reader (Perkin-Elmer). The results of the screen revealed 148 hits comprised of 140 genes and 8 microRNAs, defined according to a Z score≥2²⁰ (FIG. 1b). Here, we will only focus on gene hits, referred to as MYC synthetic lethal (MYC-SL) genes. To eliminate siRNAs that despite their differential toxicity exhibited substantial growth inhibition properties in normal cells, siRNAs with >50% reduced viability in HFF-pB were eliminated. This process left 102 MYC-SL gene hits for follow up (Table 2).

Network analysis identified known literature connections (based on Ingenuity curated database) between the “Hits” (light shading) and a pre-assembled MYC core pathway (dark shading) as shown in FIG. 5. About 50% of the MYC-SL hits had known functional connections with MYC and functionally related genes. For example TRRAP is a direct MYC binding partner that mediates recruitment of histone acetylase to selective MYC bound promoters 21 22. Several MYC-SL hits were linked to the basic transcriptional machinery (see TBP node in FIG. 5) including POLR2E, POLR21, and GTF2H4. CDK2, was also identified as MYC-SL, a finding consistent with its essential role in limiting MYC-induced senescence in a mouse model of tumorigenesis 23. Additionally, the identification of PES1 (a gene involved in ribosomal biogenesis) among MYC-SL, is consistent with the direct stimulation of ribosomal RNA synthesis by c-MYC 16 24 and by the “addiction” to elevated ribosomal function demonstrated by the suppression of MYC oncogenicity through ribosomal protein haploinsufficiency in mice 25. The broad spectrum of potential MYC-SL genes thus reflects known MYC functions linked to not only to chromatin modification (TRRAP, BRD4, CECR2,) and to ribosomal biogenesis (PES1), but also metabolism (AldoA, PDK1), DNA repair (DDB2, GTF2H4, NEIL1, POLH, RAD21), apoptosis (BNIP2, BOK, MCL1), and mitotic control (WEE1, NEK2) (see FIG. 5 and Table 2).

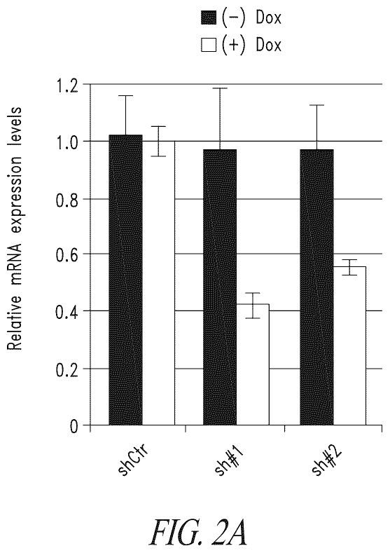

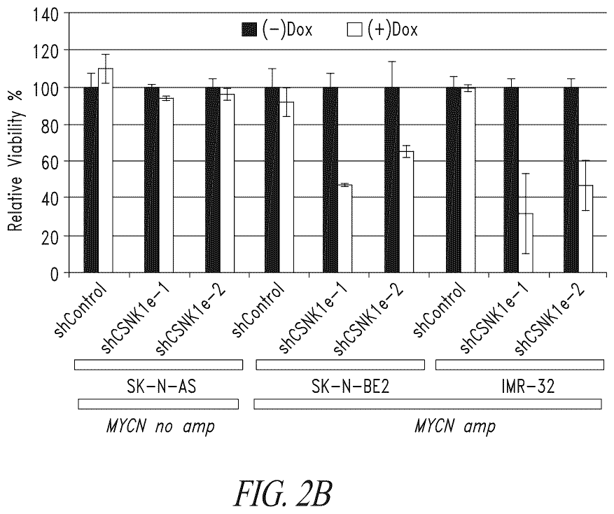

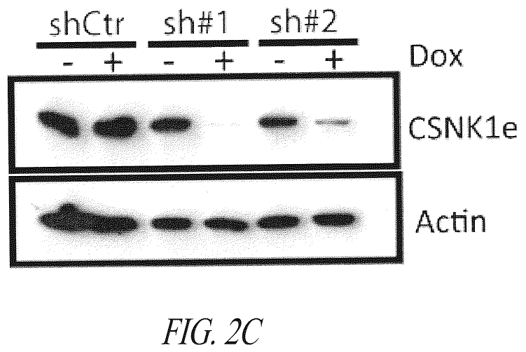

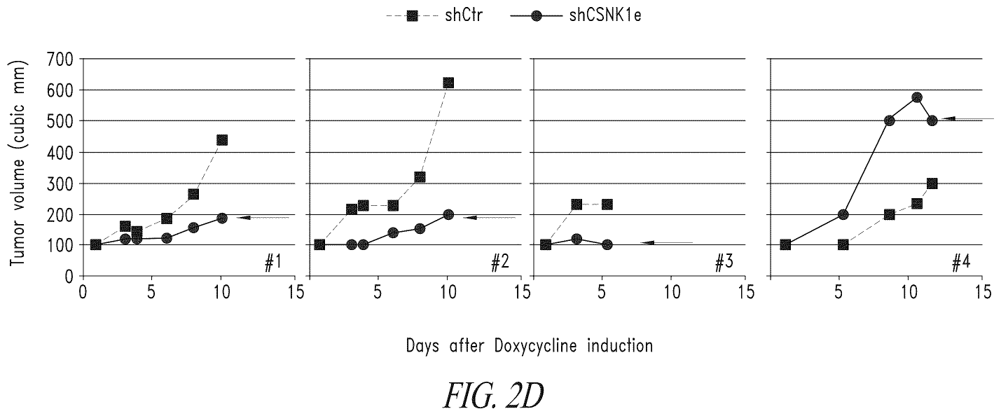

We selected 49 MYC-SL genes based upon best predicted druggability, their potential involvement in cancer pathways and their ranking in the screen in terms of differential toxicity, for follow up. Impressively, 48 out of the 49 tested genes were confirmed with more than one siRNA and in an additional matched pairs of HFFs (98% confirmation rate, see Table 1 for the list of validated and selected MYC-SL), thus highlighting the robustness of our initial screening process. Twelve MYC-SL hits, PES1, CECR2, CSNK1e, MYLK, TXK, TIE1, CDK2, PRKCL1, TRRAP, MAP3K13, NEK2 and WEE1 were assessed via stable, lentiviral-mediated shRNA knock-down, confirming their differential growth inhibition in HFF-MYC versus HFF-pB control (FIG. 1c-d, and data not shown). Examination of selective toxicity in HFF-MYC cells was carried out for 38 genes by assessing levels DNA damage and apoptosis. siRNA-mediated knock-down of twelve (25%) of the hits resulted in elevated γ-H2AX foci only in HFF-MYC but not HFF cells. This indicates that induction of DNA damage is a significant consequence of the MYC-synthetic lethal interaction (FIG. 1e for representative images and If for quantitation; summarized in Table 1). This finding is consistent with the role of MYC in promoting genomic instability²⁶ and replication associated damage due to an acceleration of S-phase^{17 27}. Additionally,³⁴ of the⁴⁸ MYC-SL genes (>70%) induced caspase-3 and 7 cleavage in HFF-MYC but not in HFF-pB upon siRNA transfection (FIG. 2g, Table 1). Importantly, our ability to recapitulate the results from the original high-throughput screen using a combination of three knockdown protocols (siRNA pools, deconvoluted siRNA pools, and lentiviral shRNAs) as well as independent assays suggests that our screening protocol was not only comprehensive but also robust and accurate in predicting MYC-SL genes.

### Casein Kinase 1 Epsilon is a MYC-SL Gene in Preclinical Models of Neuroblastoma

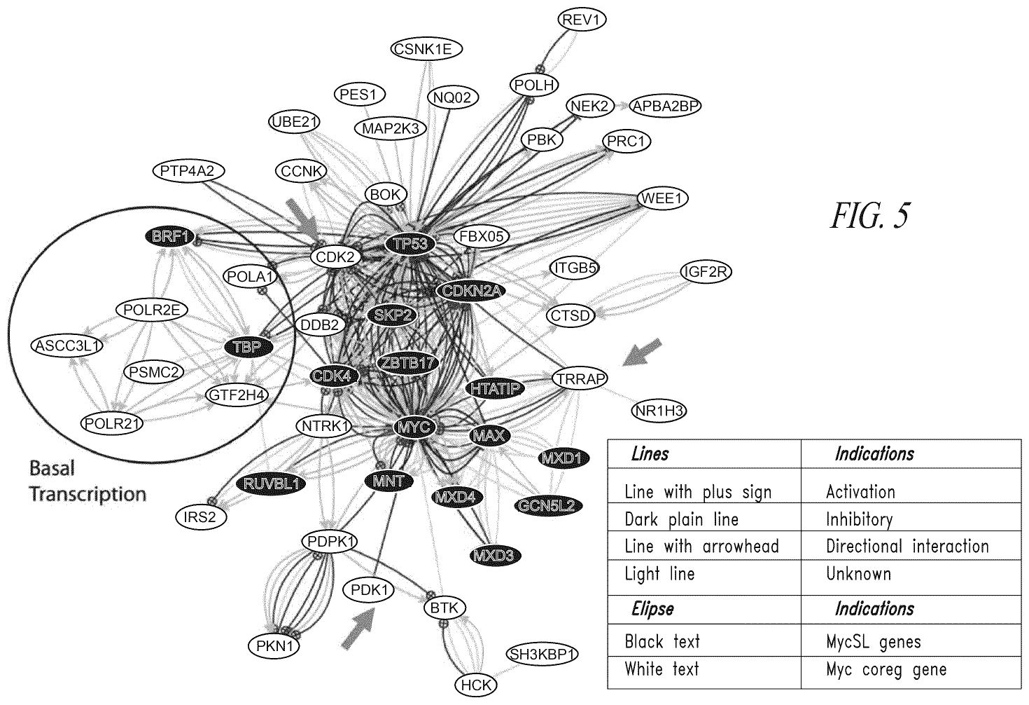

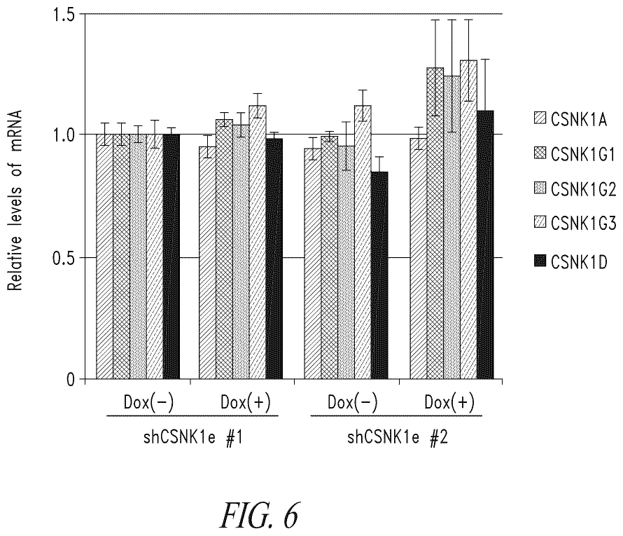

We next wished to validate the MYC-SL genes in neuroblastoma cell lines with or without MYCN amplification, as a model of MYC-driven cancer²⁸. In humans, amplification of MYCN in neuroblastoma is the strongest molecular marker of poor prognosis and is utilized for treatment stratification²⁹. The potential conservation of synthetic lethal interactions with both c-MYC and MYCN is supported by the fact that MYCN and c-MYC control a similar set of target genes and cellular phenotypes^{30 31}, and that c-MYC can replace MYCN during murine development³². We screened neuroblastoma cell lines with (IMR-32) or without (SK-N-AS) MYCN amplification with siRNAs targeting the selected 48 MYC-SL genes. 11 MYC-SL genes exhibited selective lethality in MYCN amplified neuroblastomas (indicated with shading in the first column of Table 1), indicating conserved synthetic lethal interaction with both MYC family members and in a cancer cell setting. We chose to focus on one of these genes, Casein kinase 1 epsilon (CSNK1e) for preclinical validation because siRNAs and stable knock-down had showed minimal toxicity to normal HFFs (FIG. 1), suggesting the possibility of a good therapeutic window. Moreover, pharmacologic inhibitors were readily available, enabling us to verify that blocking its enzymatic activity would mimic the effect of gene knock-down³³. We first tested the differential growth inhibition in MYCN amplified neuroblastoma cells in vitro, using conditional lentiviral vectors targeting CSNK1e with two different short hairpins (sh #1, and sh #2, FIGS. 2a, b and c). As there are six isoforms of CSNK1, the specificity of the lentiviral-expressed short hairpins was examined by assessing the relative levels of mRNA expression of the other isotypes. CSNK1e-specific short hairpins reproducibly lowered the expression of the epsilon isoform, but had no effect on the mRNA expression of the other isoforms (FIG. 6). As a pre-clinical validation model, neuroblastoma cells were transduced in vitro with either a control sh expressing lentiviral vector or shCSNK1e #1 and injected into the flanks of immunodeficient mice. Once tumors became engrafted and had reached a minimal size of ˜>100 mm³, mice were exposed to doxycycline and tumor growth was measured over time. As shown in FIG. 2d, neuroblastoma growth was significantly impaired in 3 out of 4 treated mice, validating the MYC-synthetic lethal relationship of CSNK1e knock-down in vivo.

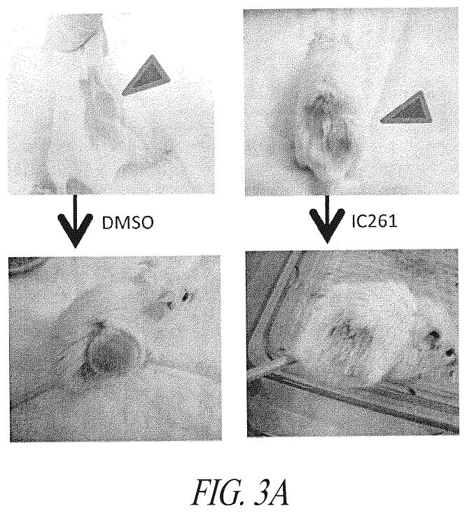

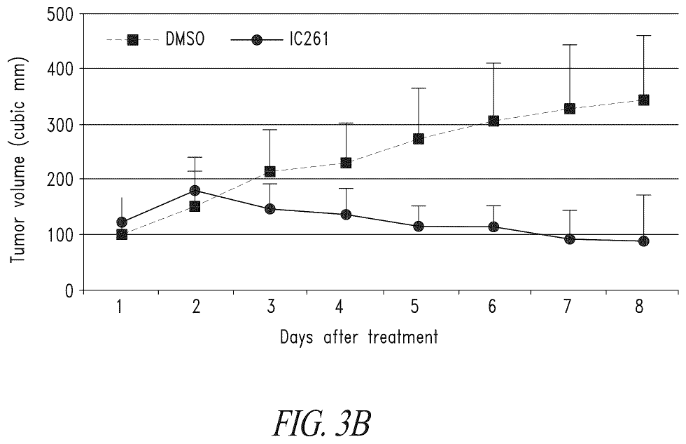

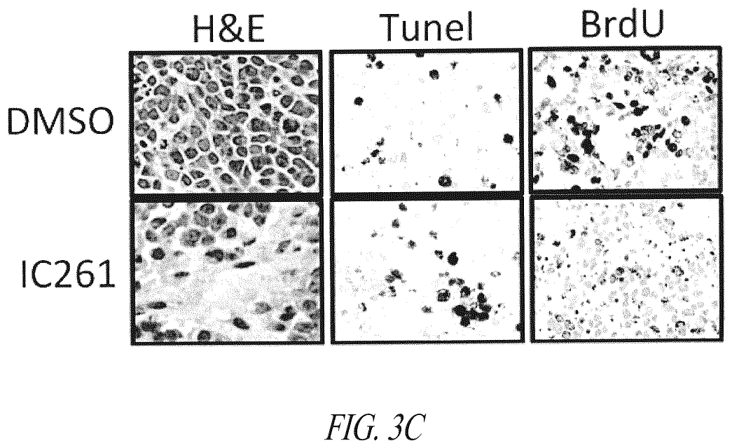

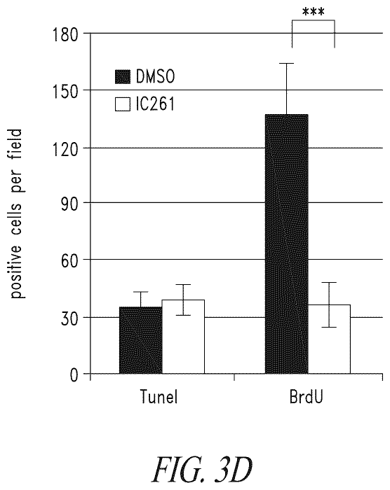

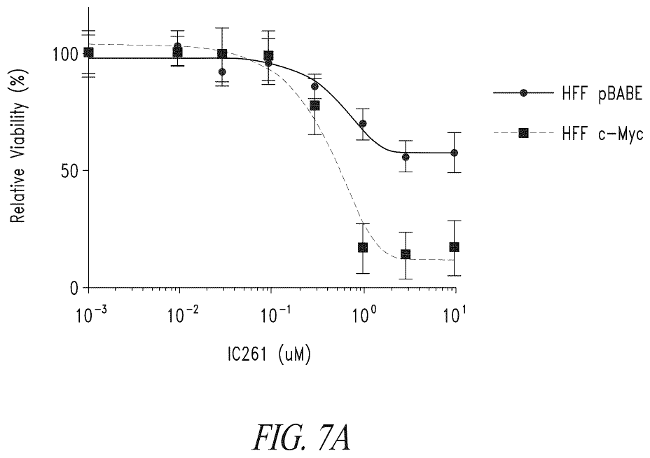

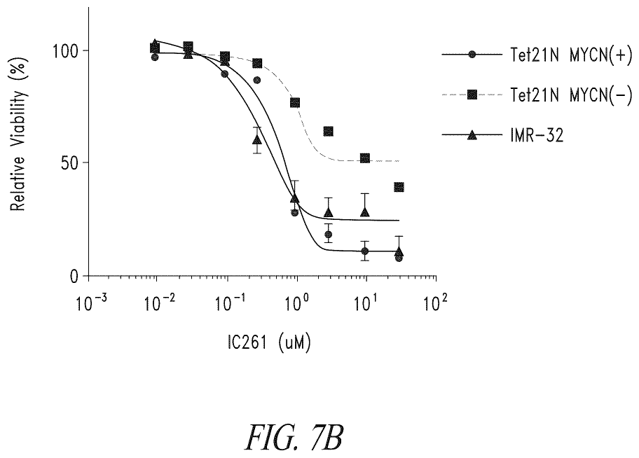

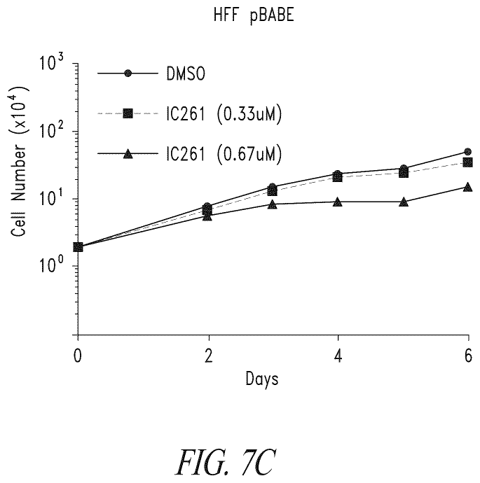

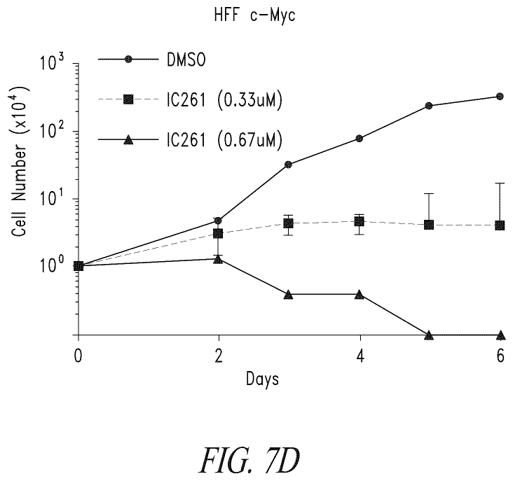

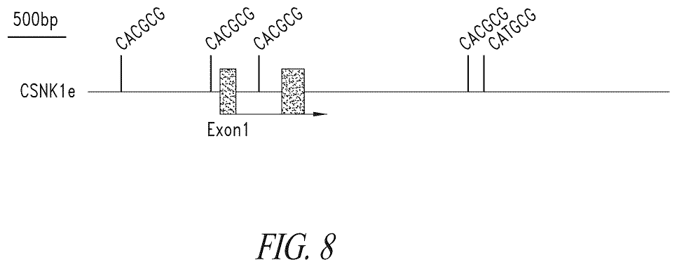

As there is strong selection to escape lentiviral-mecliated silencing of genes that are necessary for cell growth, we proceeded to evaluate a small molecule inhibitor of CSNK1e enzymatic activity, IC261³³. In vitro experiments had indicated that MYC overexpressing cells were indeed more sensitive to IC261 relative to normal or low MYC expressing cells, with >100 fold differences in IC50 (FIGS. 7a and 7b). IMR32 (MYCN+) cells were utilized as a therapeutic xenograft model as they were established in culture prior to patient chemotherapy and were highly sensitive to IC261 in vitro (FIG. 7b). A cohort of ten xenograft bearing mice was randomized into two groups with approximate equal tumor burden; one group was treated with daily subcutaneous injection of IC261 for 8 consecutive days, while the control group was treated with DMSO vehicle only. A photograph of a representative mouse from each group before and after treatment is shown in FIG. 3a. Importantly, IC261 was effective in halting tumor growth in all treated mice (FIG. 3b). Histopathological examination of the tumor tissue remaining after IC261 treatments, indicated a pronounced proliferative defect as indicated by the marked decrease in BrdU labeling, while very little apoptosis was detected via TUNEL staining (FIGS. 3c and d). This result is consistent with the observation that CSNK1e knock-down did not induce prominent caspase-3 or 7 cleavage in HFFs (FIG. 1g). Taken together, the results obtained by genetic knock-down as well as via small molecule-inhibitor validate CSNK1e as a potential therapeutic target for MYCN-driven neuroblastoma.

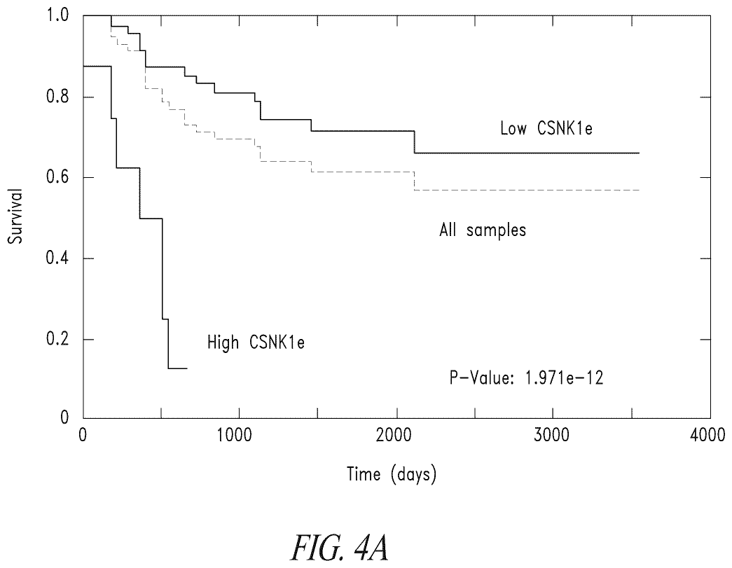

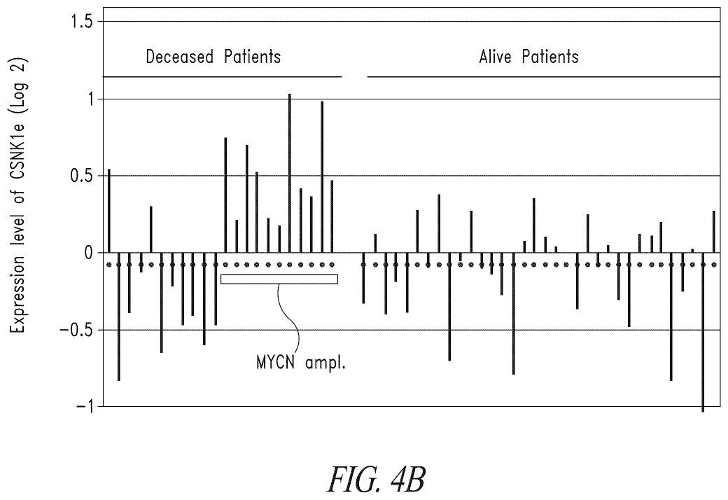

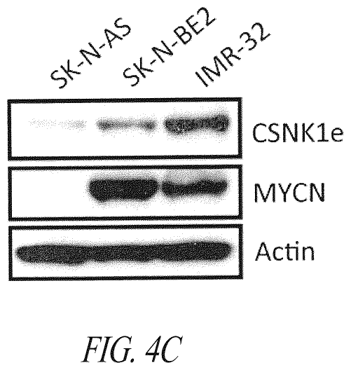

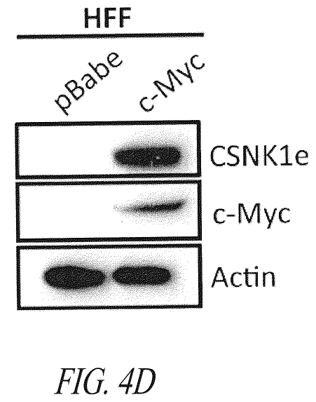

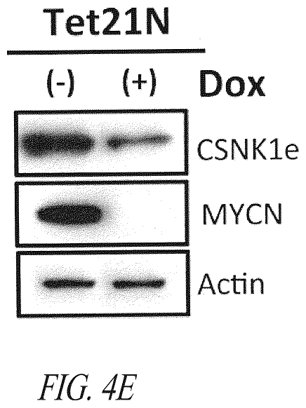

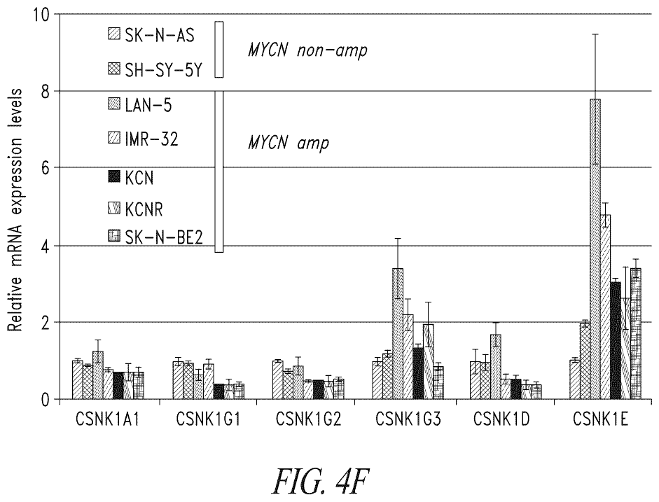

Importantly, CSNK1e expression correlates with both MYCN amplification and poor prognosis in primary neuroblastomas (FIGS. 4a and b). The correlation of high CSNK1e expression in MYCN+neuroblastoma at the protein level was confirmed in three representative cell lines (FIG. 4c) and at the RNA level in these and additional cell lines (FIG. 4f). Among the six CSNK1 isoforms, tested, epsilon, was predominantly expressed in lines with MYCN amplification³⁴ (FIG. 4f). These findings, as well as the presence of potential MYC-MAX binding sites in the promoter region of CSNK1e (FIG. 8) suggest a direct regulation of CSNK1e mRNA by c-MYC/MYCN. Consistent with this, CSNK1e is upregulated in both HFF-MYC (FIG. 4d) and upon induced MYCN expression in the neuroblastoma cell line Tet21N³⁵ (FIG. 4e). Together, these data support the model where MYC overexpression stimulates expression of CSNK1e, which is in turn required for its survival in this scenario, CSNK1e represents an “induced dependency” of MYC overexpressing cells. This finding is reminiscent of a previously identified functional dependency of MYC overexpressing cell upon one of its direct transcriptional targets, the Werner syndrome gene (WRN)³⁶.

Identifying a means to target oncogenic transcription factors as a cancer treatment remains a challenging goal, due to the non-druggability of these proteins, and their essential cellular functions in non-cancerous tissue. Here, we have identified druggable genes that are synthetically lethal in the context of high MYC expression. These genes include those known to be involved in MYC-dependent processes, as well as genes not previously identified as part of the MYC pathway. We focused on CSNK1e, a gene with no previous functional links with MYC, which we validated as a candidate therapeutic target in neuroblastoma with MYCN amplification. The potential that CSNK1e could represent a therapeutic target in other MYC-driven cancers is likely, as its expression is not restricted to HFFs or neuroblastoma, and unpublished results indicate its synthetic lethal interaction with MYC overxpression/amplification is observed in other cancer contexts.

CSNK1e has been previously implicated in the regulation of WNT and SHH signaling. Consistent with the potential for CSNK1e to affect WNT signaling, meta-analysis of gene expression in neuroblastoma tumors indicated that both Frizzeld (FZL) and its the ligand WNT10 were found elevated in MYCN+stage 4 neuroblastoma versus stage 4 MYCN-tumors, while DKK3, a WNT inhibitor, was found to be repressed (CG, unpublished observations). This finding supports the conclusion from studies in breast cancer where WNT signaling has been shown to be stimulated by MYC overexpression³⁷. Moreover, GL11, the well-studied mediator of SHH signaling, was among the bits in the HFF screen, while the receptor for SHH, smoothened (SMO), was also found elevated in MYCN+neuroblastoma by meta-analysis. Thus, it is possible that CSNK1e activity might be essential for survival of cells with MYCN amplification through its activity on both developmental pathways. During the course of this work, two publications involving functional screens also identified CSNK1e as a target to block proliferation of colon cancer and breast cancer with WNT-deregulation^{38 39}. In addition, a functional genomic screen carried out in human fibrosarcoma lines identified CSNK1e as a “hit” that differentially affected viability of transformed fibroblasts 40. Together, these findings indicate the relevance of CSNK1e in other cancer contexts and it reinforces the value of functional genomics to reveal cancer therapeutic targets, which might be missed by sequencing approaches.

In summary, here we have demonstrated an efficient pipeline, which combines the power of a robust high throughput functional genomics approach with a biological controlled cell systems, to reveal candidates for therapeutic development toward un-druggable oncogenic targets. This approach can be supplemented through the use of arrayed lentiviral libraries to enable long-term knock-down. For example, the screen did not detect the dependency of MYC upon expression of the WRN gene, likely due to the high stability of the WRN protein and mRNA and the need for HFFs to w1dergo several cell divisions under WRN depletion prior entering cellular senescence 36. Our study utilizing siRNAs has uncovered several genes that represent critical survival pathways for cancers with MYC overexpression/gene amplification. Many of these genes were not previously known to have an interaction with the MYC oncoprotein. Targeting these genes provides novel therapeutic opportunities for proliferative tissues. Inhibitors of the genes have valuable potential as cancer therapeutics. Additionally, the genes identified herein constitute biomarkers for MYC-driven cancers that can guide therapeutic choices or suggest drug combinations for maximum therapeutic effect.

TABLE 1
List of MYC-synthetic lethal genes. The effect of siRNAs pools targeting these genes
was confirmed through pool deconvolution and testing in independent HFF cell pairs. 48 out of
49 gene hits tested, which confirmed selective growth inhibition in an additional pair of matched
HFF-c-MYC and control HFF-pB and with more than one siRNA upon deconvolution of the
siRNA pool used in the screen.

| | % | % | % | | | |
|---|---|---|---|---|---|---|
| Gene | Viability | Viability | Viability | Apoptosis | H2AX | |
| Symbol | HFF-pB | HFF-MYC | PB/MYC | positive | positive | Functional Annotations |
| ALDOA | 59.31 | 13.85 | 4.28 | yes | yes | Aldolase A cleaves fructose- |
| | | | | | | 1,6-bisphosphate, glycolysis |
| ARFGEF2 | 54.66 | 3.29 | 16.62 | no | no | ADP-ribosylation factor |
| | | | | | | guanine nucleotide-exchange |
| | | | | | | factor 2 (brefeldin A- |
| | | | | | | inhibited) |
| BOK | 74.57 | 18.12 | 4.12 | yes | no | BCL2-related ovarian killer, |
| | | | | | | apoptosis in response to |
| | | | | | | DNA damage via TP53 |
| | | | | | | pathway |
| BTK | 57.41 | 8.38 | 6.85 | NT | NT | Bruton agammaglobulinemia |
| | | | | | | tyrosine kinase pre-B cell |
| | | | | | | receptor signaling and B cell |
| | | | | | | development |
| CAMK1G | 64.77 | 24.63 | 2.63 | yes | no | Calcium/calmodulin- |
| | | | | | | dependent protein kinase IG, |
| | | | | | | highly expressed in brain |
| CAMK2D | 62.00 | 17.64 | 3.51 | NT | NT | Calcium-calmodulin- |
| | | | | | | dependent protein kinase |
| | | | | | | (CaM kinase) II delta |
| CDK2 | 54.01 | 19.41 | 2.78 | yes | no | Cyclin-dependent protein |
| | | | | | | kinase 2 |
| CECR2* | 80.97 | 28.42 | 2.85 | yes | no | Cat eye syndrome |
| | | | | | | chromosome region |
| | | | | | | candidate 2, an ATPase that |
| | | | | | | interacts with SNF2L |
| | | | | | | (SMARCA1) |
| CRKRS | 59.16 | 16.71 | 3.54 | yes | no | Cdc2-related kinase arginine- |
| | | | | | | serine-rich, an SR domain- |
| | | | | | | and proline-rich region- |
| | | | | | | kinase, t phosphorylates the |
| | | | | | | C-terminal domain of RNA |
| | | | | | | polymerase II |
| CSNK1e | 76.69 | 28.59 | 2.68 | yes | no | Casein kinase 1 epsilon, |
| | | | | | | regulates Wnt receptor |
| | | | | | | signaling and circadian clock |
| CTPS | 70.57 | 19.44 | 3.63 | yes | no | CTP synthetase, inhibited by |
| | | | | | | cyclopentenyl cytosine, |
| | | | | | | increased expression in |
| | | | | | | T-ALL |
| FBXO5 | 70.44 | 25.28 | 2.79 | yes | yes | F-box containing proteins, |
| | | | | | | subunit of the SCF ubiquitin |
| | | | | | | lipase complex |
| GLI1 | 58.54 | 25.07 | 2.33 | yes | yes | Glioma-associated oncogene |
| | | | | | | homolog 1, member of the |
| | | | | | | GLI-Kruppel transcriptional |
| | | | | | | activators of the SHH |
| GTF2H4 | 73.10 | 18.01 | 4.06 | NT | NT | General transcription factor |
| | | | | | | IIH polypeptide 4 52kDa, |
| | | | | | | functions in transcription and |
| | | | | | | nucleotide excision repair |
| HCK | 67.95 | 16.98 | 4.00 | yes | no | Hematopoietic cell kinase, a |
| | | | | | | Src family tyrosine kinase |
| | | | | | | involved in signaling, |
| | | | | | | phagocytosis and cell shape |
| | | | | | | changes |
| HECTD3 | 62.93 | 28.52 | 2.21 | NT | NT | HECT domain containing |
| | | | | | | protein with similarity to |
| | | | | | | human HERC1, which acts |
| | | | | | | as a guanine-nucleotide |
| | | | | | | exchange factor for ARF1 |
| | | | | | | and Rab related proteins |
| HSD17B4 | 70.05 | 21.83 | 3.21 | NT | NT | Type IV 17 beta- |
| | | | | | | hydroxysteroid |
| | | | | | | dehydrogenase, a |
| | | | | | | peroxisomal multifunctional |
| | | | | | | enzyme involved in steroid |
| | | | | | | and bile acid metabolism |
| IGF2R | 58.76 | 15.43 | 3.81 | yes | no | Insulin-like growth factor II |
| | | | | | | receptor, a receptor tyrosine- |
| | | | | | | kinase |
| IRS2 | 72.71 | 28.46 | 2.55 | yes | no | Insulin receptor substrate 2, |
| | | | | | | mediates signal transduction |
| | | | | | | for insulin, integrin, and |
| | | | | | | cytokines, may be associated |
| | | | | | | with type 2 diabetes and |
| | | | | | | carcinoma cell invasion |
| MAP2K3 | 56.31 | 16.88 | 3.34 | yes | no | Mitogen activated protein |
| | | | | | | kinase kinase 3, |
| | | | | | | phosphorylates MAP kinase |
| | | | | | | p38, involved in stress and |
| | | | | | | inflammatory responses, |
| | | | | | | senescence, apoptosis |
| MAP2K7 | 98.97 | 43.13 | 2.29 | NT | NT | Mitogen-activated protein |
| | | | | | | kinase kinase 7,c-Jun N- |
| | | | | | | terminal kinase kinase 2 |
| MYLK | 89.77 | 13.70 | 6.55 | no | no | Myosin light polypeptide |
| | | | | | | kinase, calcium and |
| | | | | | | calmodulin-dependent |
| | | | | | | kinase, lamellipodia |
| | | | | | | protrusion/retractions |
| NEIL1 | 77.77 | 28.16 | 2.76 | yes | no | Nei endonuclease VIII-like |
| | | | | | | 1, an endonuclease with |
| | | | | | | DNA glycosylase and lyase |
| | | | | | | activities toward mismatched |
| | | | | | | or oxidized nucleotides, S |
| | | | | | | phase-specific activation |
| NEK2 | 62.97 | 27.57 | 2.28 | yes | no | NIMA-related kinase 2, |
| | | | | | | involved in centrosome cycle |
| | | | | | | during mitosis, inhibits |
| | | | | | | protein phosphatase 1 |
| PAK6 | 64.80 | 20.78 | 3.12 | NT | NT | p21(CDKN1A)-activated |
| | | | | | | kinase 6, activated by MAP |
| | | | | | | kinases, interacts with steroid |
| | | | | | | hormone receptors and |
| | | | | | | suppresses receptor-mediated |
| | | | | | | transcriptional activation |
| PCBD1 | 64.08 | 23.88 | 2.68 | yes | no | Pterin 4 alpha carbinolamine |
| | | | | | | dehydratasc, co-activator for |
| | | | | | | TCF1, altered expression is |
| | | | | | | associated with |
| | | | | | | hyperphenylalaninemia, |
| | | | | | | vitiligo, and colorectal ca. |
| PES1 | 51.38 | 14.28 | 3.60 | yes | no | Pescadillo homolog |
| | | | | | | containing BRCT domain 1, |
| | | | | | | role in transformation, rRNA |
| | | | | | | synthesis, neural crest |
| | | | | | | migration |
| PIK4CB | 58.97 | 25.54 | 2.31 | yes | no | Phosphatidylinositol 4-kinase |
| | | | | | | catalytic beta polypeptide, a |
| | | | | | | wortmannin-sensitive lipid |
| | | | | | | kinase |
| PKN1 | 86.39 | 32.82 | 2.63 | yes | no | Protein kinase, activated by |
| | | | | | | Rac, Rho and fatty acids, |
| | | | | | | stimulates phospholipase D1 |
| | | | | | | and PLC activity, regulates |
| | | | | | | G2-M and cytoskeletal |
| | | | | | | function |
| POLH | 84.32 | 28.71 | 2.94 | yes | no | DNA polymerase eta, acts in |
| | | | | | | DNA damage checkpoint by |
| | | | | | | regulating p53(TP53) |
| | | | | | | activation; gene is mutated in |
| | | | | | | xeroderma pigmentosum |
| POLR2E | 77.27 | 31.46 | 2.46 | yes | no | RNA Polymerase II DNA |
| | | | | | | directed polypeptide E 25kD, |
| | | | | | | subunit of RNA polymerases |
| | | | | | | I, II, III, involved in |
| | | | | | | transcriptional activation |
| PRC1 | 60.94 | 15.68 | 3.89 | yes | yes | Regulator of cytokinesis 1, |
| | | | | | | associates with the mitotic |
| | | | | | | spindle, required for |
| | | | | | | cytokinesis but not nuclear |
| | | | | | | division, microtubule |
| | | | | | | bundling |
| RAD21 | 62.63 | 16.73 | 3.74 | yes | yes | RAD21 homolog, sister |
| | | | | | | chromatid separation, |
| | | | | | | chromatid cohesion, G2-M |
| | | | | | | cell cycle arrest, and |
| | | | | | | cytokinesis, upregulated in |
| | | | | | | prostrate cancer, and breast |
| | | | | | | ca. |
| RAGSRF1 | 97.27 | 42.12 | 2.31 | no | no | Ras protein specific guanine |
| | | | | | | nucleotide releasing factor 1, |
| | | | | | | similar to the Saccharomyces |
| | | | | | | cerevisiae CDC25 |
| RASSF7 | 109.39 | 17.15 | 6.38 | yes | no | helix-loop-helix motif, and a |
| | | | | | | leucine zipper dimerization |
| | | | | | | motif with four heptad |
| | | | | | | repeats |
| REV1L | 76.20 | 13.26 | 5.75 | yes | no | REV1-like (yeast), a DNA |
| | | | | | | template-dependent dCMP |
| | | | | | | transferase, extends primer |
| | | | | | | strand in mutagenic |
| | | | | | | translesion DNA synthesis |
| SDC4 | 70.19 | 21.12 | 3.32 | NT | NT | Syndecan 4, regulates |
| | | | | | | inositol phospholipid binding |
| | | | | | | and signaling, protein kinase |
| | | | | | | C activation |
| SULT1A2 | 70.56 | 22.63 | 3.12 | yes | yes | Sulfotransferase cytosolic 1A |
| | | | | | | phenol-preferring member 2, |
| | | | | | | sultanates para-nitrophenol; |
| | | | | | | polymorphism associated |
| | | | | | | with early onset breast |
| | | | | | | cancer risk |
| SUV39H1 | 67.94 | 3100 | 2.19 | yes | yes | Suppressor variegation 3-9 |
| | | | | | | homolog 1, histone |
| | | | | | | methyltransferase that of |
| | | | | | | histone H3 on lysine 9, |
| | | | | | | represses transcription by |
| | | | | | | interaction with RB1 |
| TIE1 | 74.99 | 23.57 | 3.18 | yes | yes | Tyrosine kinase with |
| | | | | | | immunoglobulin-like and |
| | | | | | | EGF-like domains 1, a |
| | | | | | | putative tyrosine kinase |
| | | | | | | receptor, angiopoietin R, |
| | | | | | | upregulated in invasive |
| | | | | | | cancers |
| TRIB1 | 57.34 | 17.24 | 3.33 | yes | yes | Phosphoprotein regulated by |
| | | | | | | mitogenic pathways, a |
| | | | | | | putative kinase that interacts |
| | | | | | | with and may regulate 12- |
| | | | | | | lipoxygenase (ALOX12) |
| TRRAP | 81.13 | 31.33 | 2.59 | yes | no | ATM-related protein, a |
| | | | | | | component of a multiprotein |
| | | | | | | historic acetyltransferase |
| | | | | | | complex essential for MYC |
| | | | | | | and E2F transcription factor |
| | | | | | | pathways |
| TXK | 96.42 | 15.71 | 6.14 | no | no | Tec-Src kinases that lack the |
| | | | | | | pleckstrin domain, activates |
| | | | | | | MAPK, positively regulate |
| | | | | | | interferon gamma (IFNG) |
| | | | | | | transcription, binds PARP1 |
| UBE2I | 52.17 | 15.08 | 3.46 | yes | yes | Ubiquitin-conjugating |
| | | | | | | enzyme E2I UBC9 homolog |
| | | | | | | yeast, SUMO-1 (UBL1) |
| | | | | | | conjugating enzyme |
| WEE1 | 62.12 | 28.85 | 2.15 | yes | yes | WEE1 homolog, a tyrosine |
| | | | | | | kinase that regulates the G2 |
| | | | | | | mitotic checkpoint and |
| | | | | | | nucleocytoplasmic transport |
| WEE2 | 84.88 | 29.99 | 2.83 | yes | yes | homologue of WEE1 |
| YES1 | 68.36 | 22.16 | 3.08 | NT | NT | Yamaguchi sarcoma viral |
| | | | | | | oncogene homolog 1, a |
| | | | | | | nonreceptor protein tyrosine |
| | | | | | | kinase of the Src family, |
| | | | | | | functions downstream of |
| | | | | | | GM-CSF (CSF2) |

Shaded in “Gene Symbol” column: confirmed selective lethality in MYCN+ versus MYCN− neuroblastoma secondary screen
“no” in “Apoptosis positive” and “H2AX positive” columns: scored negative for either apoptosis or y-H2AX staining
“yes” in “Apoptosis positive” and “H2AX positive” columns: scored positive for either apoptosis or y-H2AX staining
*only 1 siRNA of the original pool was active, but confirmed by stable knock-down

TABLE 2
List of all gene hits here referred to as MYC-SL. 102 genes out of 148 hits remained
after siRNAs with percent viability of less then <50% in control HFF-pB were eliminated.
microRNAs ate also not listed here. The indicated % viability is the average of 3 replicates
and is expressed as percent viability relative to the median value of wells transfected with
an siRNA to luciferase. Hits were determined by Z score =>2, calculated as
described ²⁰ The last column refers to the ratio of percent viability of a given siRNA
pool in HFF-pB/HFF-MYC. The nucleotide sequence for each gene is hereby incorporated
by reference to the Genbank accession number, as accessed on Aug. 9, 2011
(listed in the second column).

| | Accession | Z score | % Viability | % Viability | Ratio | SEQ ID |
|---|---|---|---|---|---|---|
| Gene Symbol | number | (>than) | HFF-pB | HFF-MYC | pBabe/Myc | NO: |
| ADRBK2 | NM_005160 | 2 | 66.14 | 30.55 | 2.17 | 1 |
| ALDOA | NM_000034 | 2.5 | 59.31 | 13.85 | 4.28 | 2 |
| ALPK1 | NM_025144 | 2.5 | 59.67 | 21.80 | 2.74 | 3 |
| AMID | NM_032797 | 2 | 93.61 | 47.65 | 1.96 | 4 |
| APBA2BP | NM_031231 | 2.5 | 74.31 | 25.88 | 2.87 | 5 |
| APEG1 | NM_005876 | 2.5 | 67.41 | 20.36 | 3.31 | 6 |
| ARFGEF2 | NM_006420 | 2.5 | 54.66 | 3.29 | 16.62 | 7 |
| ASCC3L1 | NM_014014 | 2.5 | 55.94 | 18.89 | 2.96 | 8 |
| ATP5D | NM_001687 | 2 | 83.38 | 35.32 | 2.36 | 9 |
| BMPR1A | NM_004329 | 2 | 61.70 | 30.02 | 2.06 | 10 |
| BNIP2 | NM_004330 | 2 | 56.13 | 26.07 | 2.15 | 11 |
| BOK | NM_032515 | 2.5 | 74.57 | 18.12 | 4.11 | 12 |
| BRD4 | NM_014299 | 2 | 60.05 | 28.99 | 2.07 | 13 |
| BTK | NM_000061 | 2.5 | 57.41 | 8.38 | 6.85 | 14 |
| C17orf49 | BC040036 | 2.5 | 83.05 | 26.24 | 3.17 | 15 |
| C1orf117 | NM_182623 | 2.5 | 75.21 | 15.91 | 4.73 | 16 |
| CAMK1G | NM_020439 | 2.5 | 64.77 | 24.63 | 2.63 | 17 |
| CAMK2D | NM_001221 | 2.5 | 62.00 | 17.64 | 3.51 | 18 |
| CAMK2G | NM_172171 | 2 | 99.46 | 38.96 | 2.55 | 19 |
| CCNK | BC015935 | 2 | 51.64 | 21.59 | 2.39 | 20 |
| CDH5 | NM_001795 | 2.5 | 60.30 | 24.99 | 2.41 | 21 |
| CDK2 | NM_001798 | 2.5 | 54.01 | 19.41 | 2.78 | 22 |
| CECR2 | AB051527 | 2 | 80.97 | 28.42 | 2.85 | 23 |
| CPS1 | NM_001875 | 2 | 58.12 | 25.10 | 2.32 | 24 |
| CRADD | NM_003805 | 2.5 | 49.68 | 21.30 | 2.33 | 25 |
| CRKRS | NM_016507 | 2.5 | 59.16 | 16.71 | 3.54 | 26 |
| CSNK1E | NM_001894 | 2 | 76.69 | 28.59 | 2.68 | 27 |
| CTPS | NM_001905 | 2.5 | 70.57 | 19.44 | 3.63 | 28 |
| CTSD | NM_001909 | 2 | 66.42 | 36.06 | 1.84 | 29 |
| CXXC1 | NM_014593 | 2.5 | 49.66 | 18.00 | 2.76 | 30 |
| DDB2 | NM_000107 | 2 | 69.50 | 27.21 | 2.55 | 31 |
| EPNA5 | NM_001962 | 2 | 65.45 | 27.30 | 2.40 | 32 |
| FBXO5 | NM_012177 | 2.5 | 70.44 | 25.28 | 2.79 | 33 |
| GLI1 | NM_005269 | 2.5 | 58.54 | 25.07 | 2.33 | 34 |
| GNRHR | NM_000406 | 2 | 66.69 | 31.96 | 2.09 | 35 |
| GRK1 | NM_002929 | 2 | 59.11 | 29.62 | 2.00 | 36 |
| GSG2 | NM_031965 | 2 | 60.67 | 28.26 | 2.15 | 37 |
| GTF2H4 | BC004935 | 2.5 | 73.10 | 18.01 | 4.06 | 38 |
| HCK | NM_002110 | 2.5 | 67.95 | 16.98 | 4.00 | 39 |
| HECTD3 | NM_024602 | 2.5 | 62.93 | 28.52 | 2.21 | 40 |
| HPS1 | NM_000195 | 2 | 66.79 | 28.63 | 2.33 | 41 |
| HSD17B4 | NM_000414 | 2.5 | 70.05 | 21.83 | 3.21 | 42 |
| ICT1 | NM_001545 | 2.5 | 50.08 | 18.64 | 2.69 | 43 |
| IGF2R | NM_000876 | 2.5 | 58.76 | 15.43 | 3.81 | 44 |
| IRS2 | NM_003749 | 2.5 | 72.71 | 28.46 | 2.55 | 45 |
| ITGB5 | NM_002213 | 2 | 53.73 | 23.28 | 2.31 | 46 |
| KIF18A | NM_031217 | 2 | 50.87 | 21.92 | 2.32 | 47 |
| LATS1 | NM_004690 | 2 | 81.03 | 40.38 | 2.01 | 48 |
| LIMK2 | NM_005569 | 2 | 75.57 | 41.03 | 1.84 | 49 |
| MAP2K3 | NM_145110 | 2.5 | 56.31 | 16.88 | 3.34 | 50 |
| MAP2K7 | NM_145185 | 2.5 | 98.97 | 43.13 | 2.29 | 51 |
| MAP3K13 | NM_004721 | 2 | 74.85 | 29.33 | 2.55 | 52 |
| MATK | NM_139355 | 2 | 90.87 | 45.88 | 1.98 | 53 |
| MCL1 | NM_021960 | 2 | 94.93 | 52.15 | 1.82 | 54 |
| MGC11266 | NM_024322 | 2 | 56.20 | 23.98 | 2.34 | 55 |
| MLCK | NM_182493 | 2.5 | 52.37 | 13.23 | 3.96 | 56 |
| MYLK | NM_053025 | 2.5 | 89.77 | 13.70 | 6.55 | 57 |
| MYO3B | NM_138995 | 2.5 | 59.84 | 15.87 | 3.77 | 58 |
| NEIL1 | NM_024608 | 2 | 77.77 | 28.16 | 2.76 | 59 |
| NEK2 | NM_002497 | 2.5 | 62.97 | 27.57 | 2.28 | 60 |
| NQO2 | NM_000904 | 2 | 69.74 | 28.61 | 2.44 | 61 |
| NR1H3 | NM_005693 | 2 | 67.14 | 30.84 | 2.18 | 62 |
| NTRK1 | NM_002529 | 2 | 58.10 | 27.85 | 2.09 | 63 |
| PAK6 | NM_020168 | 2.5 | 64.80 | 20.78 | 3.12 | 64 |
| PBK | NM_018492 | 2 | 77.68 | 33.01 | 2.35 | 65 |
| PCBD1 | NM_000281 | 2.5 | 64.08 | 23.88 | 2.68 | 66 |
| PDK1 | NM_002610 | 2 | 69.99 | 29.57 | 2.37 | 67 |
| PES1 | NM_014303 | 2.5 | 51.38 | 14.28 | 3.60 | 68 |
| PIK4CB | NM_002651 | 2.5 | 58.97 | 25.54 | 2.31 | 69 |
| PKN1 | NM_002741 | 2 | 86.39 | 32.82 | 2.63 | 70 |
| POLA | NM_016937 | 2 | 61.21 | 23.89 | 2.56 | 71 |
| POLH | NM_006502 | 2.5 | 84.32 | 28.71 | 2.94 | 72 |
| POLR2E | NM_002695 | 2.5 | 77.27 | 31.46 | 2.46 | 73 |
| POLR2I | NM_006233 | 2 | 54.05 | 26.32 | 2.05 | 74 |
| PRC1 | NM_003981 | 2.5 | 60.94 | 15.68 | 3.89 | 75 |
| PSMC2 | NM_002803 | 2 | 60.43 | 27.13 | 2.23 | 76 |
| PTP4A2 | NM_003479 | 2 | 54.60 | 29.73 | 1.84 | 77 |
| PTPN9 | NM_002833 | 2 | 80.76 | 40.14 | 2.01 | 78 |
| RAD21 | NM_006265 | 2.5 | 62.63 | 16.73 | 3.74 | 79 |
| RASGRF1 | NM_002891 | 2.5 | 97.27 | 42.12 | 2.31 | 80 |
| RASSF7 | NM_003475 | 2.5 | 109.39 | 17.15 | 6.38 | 81 |
| REV1L | NM_016316 | 2.5 | 76.20 | 13.26 | 5.75 | 82 |
| SCYL1 | NM_020680 | 2 | 64.55 | 29.37 | 2.20 | 83 |
| SDC4 | NM_002999 | 2.5 | 70.19 | 21.12 | 3.32 | 84 |
| SH3KBP1 | NM_031892 | 2 | 61.19 | 27.82 | 2.20 | 85 |
| SLC1A4 | NM_003038 | 2 | 83.39 | 36.38 | 2.29 | 86 |
| SLC25A26 | NM_173471 | 2 | 62.05 | 34.62 | 1.79 | 87 |
| SULF2 | NM_018837 | 2.5 | 69.11 | 24.20 | 2.86 | 88 |
| SULT1A2 | NM_001054 | 2.5 | 70.56 | 22.63 | 3.12 | 89 |
| SUV39H1 | NM_003173 | 2.5 | 67.94 | 31.00 | 2.19 | 90 |
| TIE1 | NM_005424 | 2.5 | 74.99 | 23.57 | 3.18 | 91 |
| TRIB1 | NM_025195 | 2.5 | 57.34 | 17.24 | 3.33 | 92 |
| TRIP13 | NM_004237 | 2 | 71.01 | 40.01 | 1.77 | 93 |
| TRRAP | NM_003496 | 2.5 | 81.13 | 31.33 | 2.59 | 94 |
| TXK | NM_003328 | 2.5 | 96.42 | 15.71 | 6.14 | 95 |
| UBE2I | NM_003345 | 2.5 | 52.17 | 15.08 | 3.46 | 96 |
| UIP1 | NM_017518 | 2 | 66.20 | 24.65 | 2.69 | 97 |
| WEE1 | NM_003390 | 2 | 62.12 | 28.85 | 2.15 | 98 |
| WEE2 | AK131218 | 2 | 84.88 | 29.99 | 2.83 | 99 |
| WNK1 | NM_018979 | 2 | 53.61 | 26.65 | 2.01 | 100 |
| YES1 | AF119914 | 2.5 | 68.36 | 22.16 | 3.08 | 101 |

### REFERENCES

Berns, E. M. et al., c-myc amplification is a better prognostic factor than HER2/neu amplification in primary breast cancer. Cancer Res 52 (5), 1107-1113 (1992).
Blancato, J., Singh, B., Liu, A., Liao, D. J., & Dickson, R. B., Correlation of amplification and overexpression of the c-myc oncogene in high-grade breast cancer: FISH, in situ hybridisation and immunohistochemical analyses. Br J Cancer 90 (8), 1612-1619 (2004).
Sato, H., Minei, S., Hachiya, T., Yoshida, T., & Takimoto, Y., Fluorescence in situ hybridization analysis of c-myc amplification in stage TNM prostate cancer in Japanese patients. Int. J Urol 13 (6), 761-766 (2006).
Jenkins, R. B., Qian, J., Lieber, M. M., & Bostwick, D. G., Detection of c-myc oncogene amplification and chromosomal anomalies in metastatic prostatic carcinoma by fluorescence in situ hybridization. Cancer Res 57 (3), 524-531 (1997).
Kozma, L., Kiss, L, Szakall, S., & Ember, I., Investigation of c-myc oncogene amplification in colorectal cancer. Cancer Lett 81 (2), 165-169 (1994).
Park, J., Eggett, A., Caron H., Neuroblastoma: Biology, Prognosis and Treatment. Pediatric Clinics of North America 55 (1), 97-1 20 (2008).
Chen, C. H., Shen, J., Lee, W. J., & Chow, S. N., Overexpression of cyclin D1 and c-Myc gene products in human primary epithelial ovarian cancer. Int J Gynecol Cancer 15 (5), 878-883 (2005).
Takahashi, Y. et al., Amplification of c-myc and cyclin D1 genes in primary and metastatic carcinomas of the liver. Pathol Int 57 (7), 437-442 (2007).
Mitani, S. et al., Analysis of c-myc DNA amplification in non-small cell lung carcinoma in comparison with small cell lung carcinoma using polymerase chain reaction. Clin Exp Med 1 (2), 105-111 (2001).
Soucek, L. et al., Modelling Myc inhibition as a cancer therapy. Nature 455 (7213), 679-683 (2008).
Hopkins, A. L. & Groom, C. R., The druggable genome. Nat Rev Drug Discov 1 (9), 727-730 (2002).
Wang, H. et al., Improved low molecular weight Myc-Max inhibitors. Mol Cancer Ther 6 (9), 2399-2408 (2007).
Trumpp, A. et al., c-Myc regulates mammalian body size by controlling cell number but not cell size. Nature 414 (6865), 768-773 (2001).
Benanti, J. A. et al., Epigenetic down-regulation of ARF expression is a selection step in immortalization of human fibroblasts by c-Myc. Mol Cancer Res 5 (11), 1181-1189 (2007).
Benanti, J. A. & Galloway, D. A., Normal human fibroblasts are resistant to RAS-induced senescence. Mol Cell Biol 24 (7), 2842-2852 (2004).
Grandori, C. et al., c-Myc binds to human ribosomal DNA and stimulates transcription of rRNA genes by RNA polymerase 1. Nat Cell Biol 7 (3), 311-318 (2005).
Dominguez-Sola, D. et al., Non-transcriptional control of DNA replication by c-Myc. Nature 448 (7152), 445-451 (2007).
Bartz, S. R. et al., Small interfering RNA screens reveal enhanced cisplatin cytotoxicity in tumor cells having both BRCA network and TP53 disruptions. Mol Cell Biol 26 (24), 9377-9386 (2006).
Major, M. B. et al., New regulators of Wnt/beta-catenin signaling revealed by integrative molecular screening. Sci Signal 1 (45), ra 12 (2008).
Chung, N. et al., Median absolute deviation to improve hit selection for genome-scale RNAi screens. J Biomol Screen 13 (2), 149-158 (2008).
McMahon, S. B., Wood, M. A., & Cole, M. D., The essential cofactor TRRAP recruits the histone acetyltransferase hGCN5 to c-Myc. Molecular & Cellular Biology 20 (2), 556-562 (2000).
Nikiforov, M. A. et al., TRRAP-dependent and TRRAP-independent transcriptional activation by Myc family oncoproteins. Mol Cell Biol 22 (14), 5054-5063 (2002).
Campaner, S. et al., Cdk2 suppresses cellular senescence induced by the c-myc oncogene. Nat Cell Biol 12 (1), 54-59; sup pp 51-14.
Arabi, A. et al., c-Myc associates with ribosomal DNA and activates RNA polymerase I transcription. Nat Cell Biol 7 (3), 303-310 (2005).
Barna, M. et al., Suppression of Myc oncogenic activity by ribosomal protein haploinsufficiency. Nature 456 (7224), 971-975 (2008).
Ray, S. et al., MYC can induce DNA breaks in vivo and in vitro independent of reactive oxygen species. Cancer Res 66 (13), 6598-6605 (2006).
Robinson, K., Asawachaicharn, N., Galloway, D. A., & Grandori, C., c-Myc accelerates S-Phase and requires WRN to avoid replication stress. PLOS One 4 (6), e5951 (2009).
Weiss, W. A., Aldape, K., Mohapatra, G., Feuerstein, B. G., & Bishop, J. M., Targeted expression of MYCN causes neuroblastoma in transgenic mice. Embo J 16 (11), 2985-2995 (1997).
Riley, R. D. et al., A systematic review of molecular and biological tumor markers in neuroblastoma. Clin Cancer Res 10 (1 Pt 1), 4-12 (2004).
Boon, K. et al., N-myc enhances the expression of a large set of genes functioning in ribosome biogenesis and protein synthesis. EMBO J 20 (6), 1383-1393 (2001).
Mestdagh, P. et al., MYCN/c-MYC-induced microRNAs repress coding gene networks associated with poor outcome in MYCN/c-MYC-activated tumors. Oncogene 29 (9), 1394-1404.
Malynn, B. A. et al., N-myc can functionally replace c-myc in murine development, cellular growth, and differentiation. Genes Dev 14 (11), 1390-1399 (2000).
Mashhoon, N. et al., Crystal structure of a conformation-selective casein kinase-1 inhibitor. J Biol Chem 275 (26), 20052-20060 (2000).
Hanks, S. K. & Hunter, T., Protein kinases 6. The eukaryotic protein kinase superfamily: kinase (catalytic) domain structure and classification. Faseb J 9 (8), 576-596 (1995).
Lutz, W. et al., Conditional expression of N-myc in human neuroblastoma cells increases expression of alpha-prothymosin and ornithine decarboxylase and accelerates progression into S-phase early after mitogenic stimulation of quiescent cells. Oncogene 13 (4), 803-812 (1996).
Grandori, C. et al., Werner syndrome protein limits MYC-induced cellular senescence. Genes Dev 17 (13), 1569-1574 (2003).
Cowling, V. H. & Cole, M. D., Turning the tables: Myc activates Wnt in breast cancer. Cell Cycle 6 (21), 2625-2627 (2007).
Kim, S. Y. et al., CK 1 epsilon is required for breast cancers dependent on beta-catenin activity. PLOS One 5 (2), e8979.
Firestein, R. et al., CDK8 is a colorectal cancer oncogene that regulates beta-catenin activity. Nature 455 (7212), 547-551 (2008).
Yang, W. S. & Stockwell, B. R., Inhibition of casein kinase 1-epsilon induces cancer-cell-selective, PERIOD2-dependent growth arrest. Genome Biol 9 (6), R92 (2008).
Grandori, C., Mac, J., Siebelt, F., Ayer, D. E., & Eisenman, R. N., Myc-Max heterodimers activate a DEAD box gene and interact with multiple E box-related sites in vivo. EMBO Journal 15 (16), 4344-4357 (1996).

While certain embodiments of the invention have been illustrated and described, it will be appreciated that various changes can be made therein without departing from the spirit and scope of the invention.

1. A method for inhibiting the growth and/or proliferation of a MYC-driven tumor cell comprising the step of contacting the tumor cell with at least one inhibitor that inhibits the gene function of at least one of the genes listed in Table 2.

2. The method of claim 1, wherein the MYC-driven tumor cell is at least one of the following: a neuroblastoma tumor, a metastatic neuroblastoma tumor, a medulloblastoma, a lymphoma, a rhabdomyosarcoma, a melanoma, a lung cancer, a liver cancer, a breast cancer, a colon cancer, a prostate cancer, an ovarian cancer, or Burkitt's lymphoma.

3. The method of claim 1, wherein the tumor cell is contacted in vitro.

4. The method of claim 1, wherein the tumor cell is contacted in vivo in a mammalian subject.

5. The method of claim 1, wherein the inhibitor is a small molecule inhibitor that inhibits the function of the gene product.

6. The method of claim 1, wherein the inhibitor interferes with the transcription of mRNA from the gene.

7. The method of claim 1, wherein the inhibitor interferes with production or expression of the functional gene product of the gene.

8. The method of claim 1, wherein the gene is selected from the genes listed in Table 1.

9. The method of claim 1, wherein the gene is selected from the group consisting of ALDOA, CECR2, IGF2R, PAK6, PES1, RAD21, REV1L, SUV39H1, TIE1.

10. A method of treating a subject suffering from a tumor comprising MYC-driven tumor cells, comprising administering to the subject an amount of a composition comprising an inhibitor that inhibits the gene function of at least one of the genes listed in Table 2 and is effective to inhibit the growth and/or proliferation of the MYC-driven tumor cells.
